(19) United States
(12) Patent Application Publication
(10) Pub. No.: US 2025/0360478 A1
(43) Pub. Date: Nov. 27, 2025

(54) PROCESS FOR THE MANUFACTURE OF MICROCAPSULES AND MICROCAPSULES

(71) Applicant: CALYXIA SAS, Bonneuil-sur-Marne (FR)

(72) Inventors: Camille LALOUM, Ecully (FR); Florian VIGNAUD, Paris (FR); Nabila LAMAMRA, Valenton (FR); Pierre BERTRAND, Gif-sur-Yvette (FR)

(73) Assignee: CALYXIA SAS, Bonneuil-sur-Marne (FR)

(21) Appl. No.: 19/124,466

(22) PCT Filed: Oct. 25, 2023
(86) PCT No.: PCT/EP2023/079842

§ 371 Date: Apr. 25, 2025

(30) Foreign Application Priority Data
Oct. 27, 2022 (EP) 22315247.1

(51) Int. Cl. B01J 13/14

(52) U.S. Cl. CPC B01J 13/14

(57) ABSTRACT

A continuous process for preparing microcapsules having an active ingredient encapsulated in a shell of cross-linked photopolymer which includes providing a double emulsion comprising droplets of at least one active ingredient (C1) dispersed in a photopolymerizable composition C2, said droplets being dispersed in a composition C3, the compositions C2 and C3 being immiscible with each other; inducing a controlled shear rate in said double emulsion to provide a mixed double emulsion (C4); and irradiating the mixed double emulsion (C4) to prepare the microcapsules.

The present application claims priority to European Patent application 22315247.1 filed on Oct. 27, 2022, the entire contents of which is incorporated by reference into the present application.

The object of the present invention relates to a process for preparing capsules with improved retention and mechanical resistance properties, in particular an improved, continuous process for preparing such capsules. The invention also relates to the capsules as obtained as well as the use of the capsules.

A number of compounds, known as active ingredients, are added to formulated products in order to confer them with interesting beneficial application properties or to improve the performance thereof. However, in many cases, these substances react negatively with other components of the formulated product, what leads to adverse consequences on stability as well as a decline in performance levels.

The encapsulation of active ingredients represents a technique of great beneficial interest for overcoming the limitations related to performance or stability of the formulated products that contain them while also obtaining the advantageous effects derived from the active ingredients at the time of using the formulated product.

In order to completely isolate the active ingredients from the medium that contains them, it is however necessary to confer the capsules with suitable retention properties to enable retaining the active ingredients for periods of up to several years.

A very large number of capsules have been developed in order to isolate active ingredients in formulated products. These capsules are generally obtained from manufacturing methods such as spray-drying, interfacial polymerisation, interfacial precipitation, or solvent evaporation among many others.

In particular, US-A-2020129948 and US-A-2021113984 in the name of the applicant, the contents of which are incorporated by reference into the present patent application, provide capsules having very good retention properties.

The present invention now makes available a further improved process for making capsules and improved capsules which can be obtained through the process.

The invention consequently concerns a continuous process for preparing microcapsules having an active ingredient encapsulated in a shell of cross-linked photopolymer which comprises providing a double emulsion comprising droplets of at least one active ingredient C1 dispersed in a photopolymerizable composition C2, said droplets being dispersed in a composition C3, the compositions C2 and C3 being immiscible with each other;
applying a controlled shear rate to said double emulsion to provide a mixed double emulsion C4;
irradiating the mixed double emulsion C4 to prepare the microcapsules.

It has been found, surprisingly, that the process according to the invention allows for the large scale manufacture of capsules having excellent retention properties and a still improved or at least equivalent homogeneity of their characteristics such as monodispersity and wall thickness compared to known capsules. It has been found, that it is possible to improve the efficiency and the homogeneity of the photopolymerization step leading to the improved formation of the cross-linked shell of the capsules notably through higher conversion of reactive groups while substantially avoiding deterioration of the product capsules due to e.g. breakage of capsules or coalescence of droplets in the double emulsion. Retention properties of a capsule imply the ability of the capsule to retain the active ingredient until a desired external stimulus induces release of the active.

Without wishing to be bound by any theory, it is believed that the improved retention and mechanical stability of capsules arises from improvement of cross-linking during continuous processing.

For the purposes of the present invention, «continuous process » is understood to denote a process carried out in continuous mode, namely by continuously or possibly intermittently providing starting material to a reaction medium and continuously or possibly intermittently withdrawing product from the reaction medium. Preferably, the continuous process comprises continuously providing starting material to a reaction medium and continuously withdrawing product from the reaction medium.

For the purposes of the present invention, «monodisperse 141 is understood to denote with reference to a series of droplets or a series of capsules, that the standard deviation of the distribution of the diameter of said droplets or said capsules is less than 50%, in particular less than 25%, or less than 1 μm. For the purposes of the present invention, the diameter of said droplets or said capsules is determined by light scattering technique using a Mastersizer 3000 (Malvern Instruments) equipped with a Hydro SV measurement cell.

For the purpose of the present invention, “viscosity” is understood as the viscosity value measured at a shear rate of 10 s⁻¹ with a Haake RheostressTM 600 or Anton Paar MCR 92 rheometer equipped with a cone of diameter 60 mm having 2-degree angle, and a temperature control cell set at 25° C.

For the purposes of the present description, the singular includes the plural and vice versa.

In the process according the invention, the double emulsion is preferably provided through a process in accordance with US-A-2020129948 and US-A-2021113984 the contents of both of which is incorporated by reference into the present application.

In one aspect, the double emulsion can be provided through a process which comprises
a) adding, under agitation, of a composition C1, comprising at least one active ingredient, in a polymeric composition C2, the compositions C1 and C2 being immiscible with each other, the volume fraction of C1 in C2 being between 0.1 and 0.5;
the composition C2 comprising at least one monomer or polymer having an average molecular weight of less than 5000 g.mol⁻¹, at least one crosslinking agent having an average molecular weight of less than 5000 g.mol⁻¹, and optionally at least one photoinitiator having an average molecular weight of less than 5000 g.mol⁻¹ or a crosslinking catalyst having an average molecular weight of less than 5000 g.mol⁻¹;
the viscosity of the composition C2 being comprised between 500 mPa·s and 100,000 mPa·s at 25° C.;
wherein an emulsion (E1) is obtained comprising droplets of the composition C1 dispersed in the composition C2;
b) adding, under agitation, of the emulsion (E1) in a composition C3, the compositions C2 and C3 being immiscible with each other;
the viscosity of the composition C3 being comprised between 500 mPas and 100,000 mPas at 25° C. ; wherein a double emulsion (E2) is obtained comprising droplets dispersed in the composition C3;
c) applying a shear to the emulsion (E2);
wherein a double emulsion (E3) is obtained comprising size controlled droplets dispersed in the composition C3.

In the process according to the invention, the droplets of the double emulsion are preferably monodisperse.

In the process according to the invention, the induced shear rate is generally lower than 200 s⁻¹. Often the shear rate is equal to or lower than 50 s⁻¹. In the process according to the invention, the induced shear rate is generally greater than 10 s⁻¹. Often the shear rate is equal to or greater than 20 s⁻¹.The induced shear rate is generally selected to prevent coalescence of droplets

While the shear rate induced may be subject to adaptation, e.g. on account of the viscosity of the double emulsion, it is suitably selected to ensure a good photopolymerization of the shell.

In another aspect, the shear rate induced in step (b) is such that the ratio of droplets broken in step (b) is less than 0.1%, preferably less than 0.01%.

For the purposes of the present invention, the ratio of droplets is determined by optical microscopy inspection of the droplets in the double emulsion as detailed here after: The determination of droplets broken is carried out in situ, using a CSS450 Optical Rheology System from Linkam Systems. The shear rate is controlled through an Ares-G2 system from TA Instruments using the 2 Dimensional Small Amplitude Oscillatory Shear (2D-SAOS) feature.

In still another aspect, the shear rate induced in step (b) is such that the droplets of the mixed double emulsion remain monodisperse.

In a particular aspect, the shear is induced before and/or during the irradiation. For example, an initial device for inducing the shear rate can be selected which is sufficient to maintain the desired shear rate throughout the reactor. In another aspect, the shear rate is induced by an initial device for inducing the shear rate in combination with at least one subsequent device inducing an additional shear rate.

In the process according to the invention, the shear can be induced in the double emulsion, for example, using one or more devices selected from a stirrer, a vortex, a static mixer, a rotary mixer, a rotor stator mixer and an interfacial surface generator mixer.

Examples of stirrers include for example overhead mixers equipped with blades, including but not limited to helicoidal, sawtooth, cross-blade, straight-blade, pitched blade, ringed blades, anchor, propellor, radial flow, cross, paddle, centrifugal, half-moon, coil, beater, chain paddle overhead mixers and any combination thereof.

Example of vortex devices include for example tube rack vortex mixers of orbital, vertical or horizontal geometry.

Examples of static mixers include but are not limited to helical static mixers, plate-like static mixers, low pressure drop static mixers, and interfacial surface generator mixers.

Examples of rotary mixers include for example planetary mixers, orbital mixers including tank mixers for industrial scale production, and Couette mixers as described in FR 9604736.

Examples of rotor-stator mixers include commercially available devices such as for example Ross™ high shear mixers, further described in detail in for instance Håkansson, A. Rotor-Stator Mixers: From Batch to Continuous Mode of Operation—A Review. Processes 2018, 6, 32. https:/doi.org/10.3390/pr6040032.

The process according to the invention can advantageously be carried out using in-line mixers, which include but are not limited to static in-line mixers and dynamic in-line mixers.

The device can include at least one component which is directly in contact with the double emulsion. Such component may suitably be selected to provide for reduced chemical reactivity and mechanical stability thereof during the radiation step. Such component is therefore preferably made of chemically and mechanically resistant materials such as for example stainless steel, PTFE or nonreactive metals such as platinum, gold and diamond coatings.

In another aspect, the device is preferably made of a material that allows maximum dispersion of UV radiation in the double emulsion, by limiting the absorption of UV light into the device. Such materials include but are not limited to UV transparent materials such as quartz-glass or synthetic silica, borosilicates such as those disclosed in U.S. Pat. No. 5,547,904A as well as SCHOTT 8337B, 8347 and Ray Volution® D 99 glass optimized for UV transmission.

The shear rate is generally further determined by taking into account other reaction parameters such as, if appropriate, the flow rate and the geometry of the reactor.

In the process according to the invention, the irradiation is suitably carried out in one or more continuous stirred tank reactors and/or continuous flow reactors.

In a first particular aspect, the irradiation is carried out in a continuous stirred reactor wherein the double emulsion is continuously fed into the continuous stirred tank reactor and a product stream comprising microcapsules is continuously withdrawn from the continuous stirred tank reactor.

In one embodiment of the first particular aspect, a part of the product stream is recycled to the continuous stirred tank reactor. In another embodiment of the first particular aspect, the product stream comprising microcapsules is continuously introduced into at least one further irradiation step in a continuous flow reactor. In another embodiment of the first particular aspect, the product stream comprising microcapsules is continuously introduced into at least one further irradiation step in one or more continuous stirred tank reactors.

In another aspect, the irradiation is carried out in one or more continuous flow reactors. Suitably, the continuous flow reactor is equipped with at least one device for applying a shear rate such as in particular the devices described above. Preferably, the continuous flow reactor is equipped with at least one vortex and/or at least one static mixer. When a plurality of continuous flow reactors is used, said reactors can be arranged in parallel and/or in series.

When the irradiation is carried out in a flow, generally, a Reynolds number of inferior to 1 is maintained in said flow. Often the Reynolds number is equal to or lower than 0.01. Generally, the Reynolds number is greater than 0.00001.

In the process according to the invention, the irradiation can suitably be carried out in a cylindrical, flattened cylindrical, prismaticor cuboid chamber or combinations thereof.

In the process according to the invention, the average residence time in the irradiation step can be suitably adjusted in particular with the purpose of achieving a desired conversion of photopolymerizable groups, taking into consideration notably the constituents of the photopolymerizable composition C2 and the arrangement of the reactor.

The photopolymerizable composition C2 suitably comprises at least one monomer whose polymerization can be induced by radicals. A monomer comprising an acrylate and/or a methacrylate group is particularly suitable. Preferably, such monomer comprises at least 2, 3, 4, 5 or 6 acrylate and/or methacrylate groups. Alternatively, the monomer comprises another polymerizable group such as for example a mercaptoester; thiolen; siloxane; epoxy; oxetan; urethane; isocyanate; and peroxide group. Typical contents of monomer are from 50 to 99% by weight relative to the total weight of the composition C2, preferably from 60 to 95% by weight relative to the total weight of the composition C2.

In a preferred embodiment, the photopolymerizable composition C2 comprises in addition a cross-linking agent. The crosslinking agent may be suitably selected from molecules bearing at least two functional groups selected from among the group constituted of the functions: acrylate, methacrylate, vinyl ether, N-vinyl ether, mercaptoester, thiolene, siloxane, epoxy, oxetane, urethane, isocyanate, and peroxide.

By way of example of crosslinking agent, mention may be made in particular of: diacrylates, such as 1,6-hexanediol diacrylate, 1,6-hexanediol dimethacrylate, polyethylene glycol dimethacrylate, 1,9-nonanediol dimethacrylate, 1,4-butanediol dimethacrylate, 2,2-bis(4-methacryloxyphenyl)propane, 1,3-butanediol dimethacrylate, 1,10-decanediol dimethacrylate, bis(2-methacryloxyethyl)N,N′-1,9-nonylene biscarbamate, 1,4-butanediol diacrylate, ethylene glycol diacrylate, 1,5-pentanediol dimethacrylate, 1,4-phenylene diacrylate, allyl methacrylate, N,N′-methylenebisacrylamide, 2,2-bis[4-(2-hydroxy-3-methacryloxypropoxy)phenyl] propane,tetracthylene glycol diacrylate, ethylene glycol dimethacrylate, diethylene glycol diacrylate, triethylene glycol diacrylate, triethylene glycol dimethacrylate, polyethylene glycol diglycidyl ether, N,N-diallylacrylamide, 2,2-bis[4-(2-acryloxyethoxy)phenyl] propane, glycidyl methacrylate; multifunctional acrylates such as dipentaerythritol pentaacrylate, 1,1,1-trimethylolpropane triacrylate, 1,1,1-trimethylolpropane trimethacrylate, ethylenediamine tetramethacrylate, pentaerythritol triacrylate, pentaerythritol tetraacrylate;acrylates also having other reactive functional groups, such as propargyl methacrylate, 2-cyanoethyl acrylate, tricyclodecane dimethanol diacrylate, hydroxypropyl methacrylate, N-acryloxysuccinimide, N-(2-hydroxypropyl)methacrylamide, N-(3 aminopropyl)methacrylamide hydrochloride, N-(t-BOC-aminopropyl)methacrylamide, 2-aminoethyl methacrylate hydrochloride, monoacryloxyethylphosphate, o-nitrobenzyl methacrylate, acrylic anhydride, 2-(tert-butylamino)ethyl methacrylate, N,N-diallylacrylamide, glycidyl methacrylate, 2-hydroxyethyl acrylate, 2-hydroxybenzophenone, N-(Phthalimidomethyl)acrylamide, cinnamyl methacrylate. If appropriate, typical contents of cross-linking agent are from 1 to 49% by weight relative to the total weight of the composition C2, preferably from 10 to 30% by weight relative to the total weight of the composition C2.

The photopolymerizable composition C2 often comprises a photoinitiator. If appropriate, the photoinitiator is generally active in a wavelength range of from 250 to500 nm. The photoinitiator is often capable of forming free radicals which allow to induce the radical polymerization of monomers. Typical contents of photoinitiator are from 1 to 5%, preferably about 3% by weight relative to the total weight of the composition C2.

In one particular aspect, the photopolymerizable composition C2 consists of a monomer as described above, a crosslinking-agent as described above and a photoinitiator as described above, preferably in the contents indicated above.

In a particular embodiment of the process according to the invention, the average residence time in the irradiation step is generally equal to or greater than 20 s, preferably equal to or greater than 90 s. In the process according to the invention, the average residence time in the irradiation step is generally equal to or lower than 600 s, preferably equal to or lower than 300 s.

In a particular aspect of the process according to the invention, the irradiation is carried out in a flow under conditions providing a Bodenstein number of at least 50. The preferred range of Bodenstein numbers is greater than 50, preferably equal to or greater than 100, and more preferably equal to or greater than 200. Preferably, the Bodenstein number is maintained above the aforesaid value throughout the irradiation.

The Bodenstein number is a dimensionless number describing axial mixing in axial-dispersion models for flow reactors. It represents the ratio between the convective transport to the transport by axial diffusion.

It has been found that a narrow distribution of residence time in the irradiation step, as reflected by the aforementioned Bodenstein numbers applied in the afore described particular aspect of the process according to the invention, allows to assure a particularly homogeneous polymerization, which is apparent in the homogeneity of properties of the final microcapsules.

In the process according to the invention, the composition C3 has generally a viscosity of equal to or greater than 2000 m Pa*s at 25° C. Preferably this viscosity is equal to or greater than 10000 m Pa*s at 25° C. In the process according to the invention, the composition C3 has generally a viscosity of equal to or lower than 100000 m Pa*s at 25° C. Preferably this viscosity is equal to or lower than 50000 m Pa*s at 25° C.

In the process according to the invention, the photopolymerizable composition C2 is generally photopolymerizable in the wavelength range of 100-500 nm, usually 200-450 nm, preferably 300-450 nm. In another embodiment, the photopolymerizable composition C2 is generally photopolymerizable in the wavelength range of 100-400 nm, preferably 300-400 nm.

In the process according to the invention, the composition C3 has generally an absorbance of 0.5% to 30% in the wavelength range of 100-400 nm.

In the process according to the invention, the irradiation is generally carried out using at least one source of radiation emitting radiation in the wavelength range of 100-500 nm, usually 200-450 nm, preferably 300-400 nm which irradiates the mixed double emulsion through a barrier displaying transmittance at the wavelength of emission. In this case, the source of radiation preferably emits perpendicularly to the barrier located closest to the radiation source. The source of radiation may however also be positioned to emit in other directions as long as sufficient radiation is provided to the mixed double emulsion. For example such direction can be between a perpendicular and a parallel orientation between the source of radiation and the barrier.

In the process according to the invention, the thickness of the mixed double emulsion in the direction of propagation of the radiation is generally from 1 mm to 20 cm, preferably from 5 mm to 5 cm.

The source of radiation may be placed inside the reactor, for example in the center of an irradiation chamber or at an edge of an irradiation chamber. The source of radiation may also be placed outside the reactor. In some aspects, multiple sources of radiation may be placed inside and/or outside the reactor.

The barrier material can be comprised of a material that allows maximum transmittance of UV radiation to the emulsion, by limiting the absorption of UV light into the mixer. Such materials include but are not limited to UV transparent materials such as quartz-glass or synthetic silica, borosilicates such as those disclosed in U.S. Pat. No. 5,547,904A as well as SCHOTT 8337B, 8347 and Ray Volution® D 99 glass optimized for UV transmission.

In the process according to the invention the active ingredient can be suitably selected from
for example:
a crosslinking agent, a hardener, an organic or metal catalyst (such as an organometallic or inorganometallic complex of platinum, palladium, titanium, molybdenum, copper, zinc) used for polymerising polymer-, elastomer-, rubber-, paint-, adhesive-, sealant-, mortar-, varnish-, or coating formulations;
a dye or pigment intended for elastomer-, paint-, coating-, adhesive-, sealant-, mortar-, or paper formulations;
a fragrance (in accordance with the list of molecules established by the International Fragrance Association (IFRA) and available on the website www.ifraorg.org) intended for detersive products such as cleaning/washing products, home care products, cosmetic and personal care products, textiles, paints, coatings; an aroma/flavouring agent, a vitamin, an amino acid, a protein, a lipid, a probiotic, an antioxidant, a pH corrector, a preservative for food compounds and animal feed; a softener, a conditioning agent for detersive products, cleaning/washing products, cosmetics and personal care products. In this regard, the active agents that may be used are for example as listed in the US patents U.S. Pat. No. 6,335,315 and U.S. Pat. No. 5,877,145;
an anti-discolouration or anti-fading agent (such as an ammonium derivative), an antifoaming agent (such as an alcohol ethoxylate, an alkylbenzene sulfonate, a polyethylene ethoxylate, an alkylethoxysulfate or alkylsulfate) intended for detersive products and cleaning/washing products and home care products;
a brightening agent, also referred to as a colour activating agent (such as a stilbene derivative, a coumarin derivative, a pyrazoline derivative, a benzoxazole derivative, or a naphthalimide derivative) intended for detersive products, cleaning/washing products, cosmetics and personal care products;
a biologically active compound such as an enzyme, a vitamin, a protein, a plant extract, an emollient agent, a disinfecting agent, an antibacterial agent, an anti-UV agent, a medicament intended for cosmetic and personal care products, and textiles. Among these biologically active compounds the following may be mentioned: vitamins A, B, C, D and E, para-aminobenzoic acid, alpha hydroxy acids (such as glycolic acid, lactic acid, malic acid, tartaric acid, or citric acid), camphor, ceramides, polyphenols (such as flavonoids, phenolic acid, ellagic acid, tocopherol, ubiquinol), hydroquinone, hyaluronic acid, isopropyl isostearate, isopropyl palmitate, oxybenzone, panthenol, proline, retinol, retinyl palmitate, salicylic acid, sorbic acid, sorbitol, triclosan, tyrosine;
a disinfecting agent, an antibacterial agent, an anti-UV agent, intended for paints and coatings;
a fertiliser, a herbicide, an insecticide, a pesticide, a fungicide, a repellent, or a disinfecting agent intended for agrochemical products;
a fire resistant agent, also known as a flame retarding agent, (for example a brominated polyol such as tetrabromobisphenol A, a halogenated or non-halogenated organophosphorus compound, a chlorinated compound, an aluminum trihydrate, an antimony oxide, a zinc borate, a red phosphorus, a melamine, or a magnesium dihydroxide) intended for use in plastic materials, coatings, paints, and textiles; a photonic crystal or a photochromophore intended for use in paints, coatings, and in polymer materials that form curved and flexible screens;
a product known to the person skilled in the art under the accepted nomenclature Phase Change Materials (PCMs) that is capable of absorbing or releasing so-called ‘latent’ heat when going through a change in a phase, intended for the storage of energy. Examples of PCMs and the applications thereof are described in “A review on phase change energy storage: materials and applications”, Farid et al., Energy Conversion and Management, 2004, 45 (9-10), 1597-1615. By way of examples of PCMs, mention may be made of molten salts of aluminum phosphate, ammonium carbonate, ammonium chloride, cesium carbonate, cesium sulfate, calcium citrate, calcium chloride, calcium hydroxide, calcium oxide, calcium phosphate, calcium saccharate, calcium sulfate, cerium phosphate, iron phosphate, lithium carbonate, lithium sulfate, magnesium chloride, magnesium sulfate, manganese chloride, manganese nitrate, manganese sulfate, potassium acetate, potassium carbonate, potassium chloride, potassium phosphate, rubidium carbonate, rubidium sulfate, disodium tetraborate, sodium acetate, sodium bicarbonate, sodium bisulfate, sodium citrate, sodium chloride, sodium hydroxide, sodium nitrate, sodium percarbonate, sodium persulfate, sodium phosphate, sodium propionate, sodium selenite, sodium silicate, sodium sulfate, sodium tellurate, sodium thiosulfate, strontium hydrophosphate, zinc acetate, zinc chloride, sodium thiosulfate, paraffinic hydrocarbon waxes, polyethylene glycols.

In the process according to the invention, the photopolymer forming the shell of the microcapsules is generally selected from polyethers, polyesters, polyurethanes, polyureas, polyethylene glycols, polypropylene glycols, polyamides, polyacetals, polyimides, polyolefins, polysulfides, and polydimethylsiloxanes, said polymers bearing at least one reactive function selected from the group consisting of acrylate; methacrylate; vinyl ether; N-vinyl ether; mercaptoester; thiolen; siloxane; epoxy; oxetan; urethane; isocyanate; and peroxide.

In the process according to the invention, the mean diameter of the microcapsules produced is generally between 1 μm and 30 μm.

In the process according to the invention, the microcapsules produced have usually a solid enveloping shell. The thickness of said shell is preferably between 0.2 μm and 8 μm.

The invention also concerns a series of solid microcapsules, in which each microcapsule includes:
a core comprising a composition C1 as defined in claim 1; and
a solid enveloping shell that completely encapsulates at its periphery the core, the said solid enveloping shell comprising pores that are less than 1 nm in size;
in which the mean diameter of the said microcapsules is between 1 μm and 30 μm, the thickness of the solid enveloping shell is between 0.2 μm and 8 μm, the standard deviation of the distribution of the diameter of microcapsules is less than 50%, or less than 1 μm and the conversion of reactive groups of the photopolymerizable composition C2 is at least 80%, preferably at least 90%. Preferably the distribution of conversion rates in a series of microcapsules according to the invention has a standard deviation which is not greater than 5%.

It has been found that the series of microcapsules according to the invention having a high and homogeneous conversion rate of reactive groups allow to achieve particularly interesting mechanical stability and release properties of the microcapsules.

Consequently, in a particular aspect, the invention concerns a series of microcapsules, each microcapsule having a core containing an active ingredient solid enveloping shell obtained by conversion of reactive groups, the thickness of said shell being between 0.2 μm and 8 μm, said microcapsules having a mean diameter between 1 μm and 30 μm and the standard deviation of the distribution of the diameter of microcapsules being less than 50%, or less than 1 μm, wherein the conversion of reactive groups is at least 80%, preferably at least 90% and the distribution of conversion rates has a standard deviation not greater than 5%.

The conversion of reactive groups can be determined by the monitoring of the disappearance of one band representative of a functional group under FTIR, the absorption of IR bands being proportional to the amount of the functional group, therefore the reduction of peak height corresponds to the reduction of the amount of the functional group, further indicating successful polymerization. The standard method of doing this is comparison of the FTIR absorption of the emulsion before and after photopolymerization. For the purpose of the present invention this can be done using the method disclosed in Barszczewska-Rybarek, Materials 2019, 12 (24), 4057.

The different series of microcapsules according to the invention can be obtained by the process according to the invention.

The invention also concerns the use of the microcapsules in accordance with the invention for the delivery of an active ingredient.

To the extent that there would be any inconsistencies between any document incorporated by reference and the present description, the present description shall take precedence.

The examples here after are intended to illustrate the invention without however limiting it.

### EXAMPLES

### Example 1

### Preparation of Double Emulsion According to US2021113984

### [Step a): Creation of the Core of the Capsules (Dispersion of Particles—Composition C1b)

TABLE 1

| Weight | (g) | % |
|---|---|---|
| Composition C1a | | |
| Solvesso 200 ND | 14 | 40 |
| Saturated triglyceride wax | 6 | 17.1 |
| Suppocire DM wax, | | |
| Gattefoss e) | | |
| Composition B | | |
| Dispersant (Tween 80, | 2 | 5.7 |
| Sigma Aldrich) | | |
| Deionized water | 13 | 37.2 |
| Total | 35 | 100 |

The composition C1a is placed in a bath thermostated at 35° C. and stirred at 500 rpm until complete dissolution of the wax. Composition B is placed in a bath thermostated at 35° C. and stirred at 200 rpm until complete homogenization. The composition C1a is then added to the composition B dropwise under stirring at 2000 rpm, still at 35° C. The mixture is stirred at 2000 rpm for 5 minutes and then sonicated (Vibra-cell 75042, Sonics) for 20 minutes (pulse 5 s/2 s) at 30% amplitude. If the temperature exceeds 35° C. during sonication, the mixture is cooled by ice After cooling, 1.05 g of modified polyethylene glycol gelling agent (Aculyn 44N, Dow) is added to the mixture under stirring at 500 rpm until gelation. The composition C1b is thus obtained.

### Step b): Preparation of the First Emulsion (E1)

TABLE 2

| | Components | Weight (g) | % Total |
|---|---|---|---|
| Composition C1b | | 3 | 30 |
| Composition C2 | | 7 | 70 |
| | CN981 (urethane | 6.09 | |
| | acrylateoligomer, | | |
| | Sartomer) | | |
| | HDDA | 0.7 | |
| | (hexane-1,6-diol di- | | |
| | acrylate, Sartomer) | | |
| | Darocur 1173 | 0.21 | |
| | (photoinitiateur, | | |
| | BASF) | | |
| TOTAL | | 10 | 100 |

The composition C1 is added dropwise to the composition C2 under stirring at 2000 rpm, at room temperature T

### Step c): Preparation of the Second Emulsion (E2)

TABLE 3

| | | Weight (g) | % total |
|---|---|---|---|
| First emulsion (E1) | | 5 | 5 |
| Composition C3 | Modified | 2.85 | 2.85 |
| | polyethylene glycol | | |
| | gelling agent | | |
| | (Aculyn 44N, Dow) | | |
| | Deionized water | 92.15 | 92.15 |
| | Total | 100 | 100 |

The composition C3 is stirred at 1000 rpm until complete homogenization. The first emulsion (E1) is then added dropwise to the composition C3 under stirring at 1200 rpm, at room temperature T

### Step d): Refining in Size of the Second Emulsion

The second polydisperse emulsion obtained in the preceding step is stirred at 1200 rpm for 10 minutes, at a temperature Td=20° C. A monodisperse emulsion (E3) is thus obtained.

### Example 2

### Photopolymerization in Accordance With the Invention

A volume of 3000 mL of double emulsion E3 is prepared as described in example 1 here before. A quartz flask having a useful volume of 1000 mL, equipped with a feed line, a withdrawal line and a stirring device, imparting a shear rate of 70 s⁻¹ is filled with double emulsion E3 obtained as described here before, having a transmittance of 0.9 and a viscosity of 5 000 mPa*s. Stirring is initiated to provide a mixed double emulsion E4 and a UV lamp emitting at 365 nm having a maximum light intensity of 1 W/cm2 arranged perpendicularly to a wall of the flask is turned on. A flow of 300 mL/min of mixed polymerized double emulsion is continuously withdrawn through the withdrawal line while fresh double emulsion is fed through the feed line at the same rate

The obtained microcapsules are monodisperse. Substantially no coalescence of droplets is observed. The conversion of reactive groups is at least 80%

### Example 2a

The process of example 2 is carried out but in addition the mixed double emulsion withdrawn is allowed to flow through a quartz tube having a diameter of 5 cm irradiated by a second UV lamp emitting at 365 nm having a maximum light intensity of 1 W/cm2.The obtained microcapsules are monodisperse. Substantially no coalescence of droplets is observed. The conversion of reactive groups is at least 90%

### Example 2b

The process of example 2 is carried out but in addition the flask is equipped with a recycle line whereby 50% of the stream withdrawn is recycled. The feed rate of double emulsion E3 is adjusted correspondingly. The obtained microcapsules are monodisperse. Substantially no coalescence of droplets is observed. The conversion of reactive groups is at least 80%.

### Example 2c

The double emulsion E3 is continuously introduced into a tube equipped with a static mixer imparting a shear rate of 70 s⁻¹. The Reynolds number is 0.1. The mixed double emulsion is then fed at a rate of 300 ml/min into the feed line of the flask and the irradiation is carried out as described in example 2. The obtained microcapsules are monodisperse. Substantially no coalescence of droplets is observed. The conversion of reactive groups is at least 80%

### Example 2d

The process of Example 2c is carried out but the mixed double emulsion is irradiated in a quartz tube in accordance with example 2a instead of the flask. The obtained microcapsules are monodisperse. Substantially no coalescence of droplets is observed. The conversion of reactive groups is at least 80%

### Comparative Example 1

200 ml of the double emulsion (E3) obtained in example 1 is poured into a 500 ml beaker and irradiated for 15 minutes with the aid of a UV light source (Dymax LightBox ECE 2000) having a maximum light intensity of 1 W/cm2 at a waveform length of 365 nm. The obtained microcapsules are substantially monodisperse but some coalescence of droplets is observed. The conversion of reactive groups is below 75%.

### Example 3

### Preparation of a Double Emulsion According to US2020129948

### Step a): Preparation of the First Emulsion (E1)

TABLE 4

| | Raw materials | % in C2 | % in E1 |
|---|---|---|---|
| Composition C1 | Parafin oil | | 30 |
| Composition C2 | CN 1963 (aliphatic | 80 | 70 |
| | urethane acrylate, | | |
| | Sartomer) | | |
| | SR 399 | 17 | |
| | (polymerizing agent, | | |
| | dipentaerythritol | | |
| | pentacrylate, | | |
| | Sartomer) | | |
| | Darocur 1173 | 3 | |
| | (photoinitiator, | | |
| | BASF) | | |
| Total | | 100 | 100 |

The composition C2 has the following characteristics:

CN component 1963 has 2 reactive acrylate functions per molecule and an average molecular weight of less than 5,000 g/mol. The crosslinking agent SR 399 has 5 reactive acrylate functions per molecule and a molecular weight of 524.5 g/mol. The Darocur 1,173 photoinitiator has no reactive functions and its molecular weight is 164 g/mol. The composition C1 is added dropwise to the composition C2 with stirring at 2000 rpm with a ratio of 3:7.The first emulsion (E1) is thus obtained.

### Step b): Preparation of the Second Emulsion (E2)

TABLE 5

| | Raw Materials | % |
|---|---|---|
| First Emulsion (E1) | | 5 |
| Composition C3 | Sodium Alginate | 9.5 |
| | (Sigma Aldrich) | |
| | Deionized water | 85.5 |

| | |
|---|---|
| Total | 100 |

The composition C3 is stirred at 1000 rpm until complete homogenization and then left to stand for one hour at room temperature. The first emulsion (E1) is then added dropwise to the composition C3 with stirring at 1000 rpm. This gives the second emulsion (E2).

### Step c): Refining in Size of the Second Emulsion

The second polydisperse emulsion (E2) obtained in the previous step is stirred at 1000 rpm for 10 minutes. A monodisperse emulsion (E3) is thus obtained.

### Example 4

### Photopolymerization in Accordance With the Invention

A volume of 3000 mL of double emulsion E3 is prepared as described in example 3 here before. A quartz flask having a useful volume of 1000mL, equipped with a feed line, a withdrawal line and a stirring device, imparting a shear rate of 70 s⁻¹, is filled with double emulsion E3 obtained as described here before, having a transmittance of 0.9 and a viscosity of 5 000 mPa*s. Stirring is initiated to provide a mixed double emulsion E4 and a UV lamp emitting at 365 nm having a maximum light intensity of 1 W/cm2 arranged perpendicularly to a wall of the flask is turned on. A flow of 300 mL/min of mixed polymerized double emulsion is continuously withdrawn through the withdrawal line while fresh double emulsion is fed through the feed line at the same rate. The obtained microcapsules are monodisperse. Substantially no coalescence of droplets is observed. The conversion of reactive groups is at least 80%

### Example 4a

The process of example 4 is carried out but in addition the mixed double emulsion withdrawn is allowed to flow through a quartz tube having a diameter of 5 cm irradiated by a second UV lamp emitting at 365 nm having a maximum light intensity of 1 W/cm2, the quartz tube further comprising a rotostator mixer. The obtained microcapsules are monodisperse. Substantially no coalescence of droplets is observed. The conversion of reactive groups is at least 90%.

### Example 4b

The process of example 4 is carried out but in addition the flask is equipped with a recycle line whereby 50% of the stream withdrawn is recycled. The feed rate of double emulsion E3 is adjusted correspondingly. The obtained microcapsules are monodisperse. Substantially no coalescence of droplets is observed. The conversion of reactive groups is at least 80%

### Example 4c

The double emulsion E3 is continuously introduced n into a tube equipped with a static mixer imparting a shear rate of 70 s⁻¹. The Reynolds number is 0.1.The mixed double emulsion is then fed at a rate of 300 ml/min into the feed line of the flask and the irradiation is carried out as described in example 2. The obtained microcapsules are monodisperse. Substantially no coalescence of droplets is observed. The conversion of reactive groups is at least 80%

### Example 4d

The process of Example 4c is carried out but the mixed double emulsion is irradiated in a quartz tube in accordance with example 4a instead of the flask. The obtained microcapsules are monodisperse. Substantially no coalescence of droplets is observed. The conversion of reactive groups is at least 80%

### Comparative Example 2

200 ml of the double emulsion (E3) obtained in example 3 is poured into a 500 ml beaker and irradiated for 15 minutes with the aid of a UV light source (Dymax LightBox ECE 2000) having a maximum light intensity of 1 W/cm2 at a waveform length of 365 nm. The obtained microcapsules are substantially monodisperse but some coalescence of droplets is observed. The conversion of reactive groups is below 75%.

1. A continuous process for preparing microcapsules having an active ingredient encapsulated in a shell of cross-linked photopolymer which comprises providing a double emulsion comprising droplets of at least one active ingredient (C1) dispersed in a photopolymerizable composition C2, said droplets being dispersed in a composition C3, the compositions C2 and C3 being immiscible with each other; inducing a controlled shear rate in said double emulsion to provide a mixed double emulsion (C4); and irradiating the mixed double emulsion (C4) to prepare the microcapsules.

2. The process according to claim 1 wherein the induced shear rate is lower than 200 s⁻¹, preferably from 50 to 200 s⁻¹.

3. (canceled)

4. The process according to claim 1, wherein the droplets of the double emulsion are monodisperse and the shear rate induced is such that the droplets of the mixed double emulsion remain monodisperse.

5. The process according to claim 1, wherein the shear rate is induced using a stirrer, a vortex, a static mixer, a rotary mixer or a rotor-stator mixer.

6.-11. (canceled)

12. The process according to claim 1, wherein the shear rate is induced using an in-line mixer.

13. (canceled)

14. The process according to claim 1, wherein the irradiation is carried out in one or more continuous stirred tank reactors and/or continuous flow reactors.

15.-20. (canceled)

21. The process according to claim 14, wherein the irradiation is carried out in one or more continuous flow reactors.

22. The process according to claim 21 wherein the continuous flow reactor is equipped with at least one device for applying a shear rate.

23.-27. (canceled)

28. The process according to claim 21, wherein a plurality of continuous flow reactors is used and said reactors are arranged in parallel and/or in series.

29. (canceled)

30. The process according to claim 1, wherein the photopolymerizable composition C2 comprises or consists of a monomer whose polymerization can be induced by radicals, an optional crosslinking-agent and a photoinitiator

31.-32. (canceled)

33. The process according to claim 30, wherein the composition C2 comprises or consists of 50% to 99%, preferably 60% to 95% of monomer, 1% to 5%, preferably about 3% of photoinitiator and optionally 1% to 49%, preferably 10% to 30% of cross-linking agent, all percentages being by weight relative to the total weight of composition C2.

34. The process according to claim 1, wherein the composition C3 has an absorbance of 0.5 to 3 in the wavelength range of from 100 to 400 nm.

35.-38. (canceled)

39. The process according to claim 1, wherein the shear rate is induced before and/or during the irradiation.

40. The process according to claim 1, wherein the irradiation is carried out in a flow wherein a Reynolds number inferior to 1, preferably from 0.00001 to 0.01 is maintained in said flow.

41. The process according to claim 1, wherein the irradiation is carried out in a flow under conditions providing a Bodenstein number of at least 50.

42. The process according to claim 1, wherein the average residence time in the irradiation step is from 20 to 600 s.

43. The process according to claim 1, wherein the thickness of the mixed double emulsion in the direction of propagation of the radiation is 1 mm to 20 cm.

44. The process according to claim 1, wherein the irradiation is carried out in a cylindrical, flattened cylindrical, prismatic or cuboid chamber or combinations thereof.

45.-47. (canceled)

48. A series of microcapsules, each microcapsule having a core containing an active ingredient solid enveloping shell obtained by conversion of photoreactive groups, the thickness of said shell being between 0.2 μm and 8 μm, said microcapsules having a mean diameter between 1 μm and 30 μm and the standard deviation of the distribution of the diameter of microcapsules being less than 50%, or less than 1 μm wherein the conversion of photoreactive groups is at least 80% and the distribution of conversion rates has a standard deviation not greater than 5%.

49. The plurality of microcapsules according to claim 48, wherein the conversion of photoreactive groups is at least 90%.

50. (canceled)
